(19) United States
(12) Patent Application Publication
(10) Pub. No.: US 2016/0256407 A1
(43) Pub. Date: Sep. 8, 2016

(54) ENERGY PATCH

(71) Applicant: XEP, INC., Encinitas, CA (US)

(72) Inventors: Servet Buyuktimkin, San Diego, CA (US); Nadir Buyuktimkin, San Diego, CA (US); James L. Yeager, Lake Forest, IL (US)

(21) Appl. No.: 15/027,910

(22) PCT Filed: Oct. 10, 2014
(86) PCT No.: PCT/US14/60090

§ 371 Date: Apr. 7, 2016

Related U.S. Application Data
(60) Provisional application No. 61/889,767, filed on Oct. 11, 2013.

(51) Int. Cl. A61K 9/70; A61K 31/522; A61K 36/185; A61K 36/77

(52) U.S. Cl. CPC A61K 9/7061; A61K 36/77; A61K 31/522; A61K 36/185

(57) ABSTRACT

A transdermal patch suitable for releasing a stimulant such as caffeine for an extended time period utilizes a pressure sensitive acrylic adhesive matrix constituted by an acrylic adhesive having hydroxy groups and an acrylic adhesive without hydroxy, groups, preferably in a respective weight ratio of about 2 to about 3. A suitable caffeine source is caffeine-fortified guarana seed extract, caffeine, a caffeine salt, and the like.

### CROSS-REFERENCE TO RELATED APPLICATION

This application claims the benefit of U.S. Provisional Application No. 61/889,767, filed on Oct. 11, 2013, which is incorporated herein by reference in its entirety.

### FIELD OF INVENTION

This invention relates to transdermal delivery devices for biologically active ingredients, in particular to transdermal patches that release a xanthine composition, e.g., caffeine, over an extended time period.

### BACKGROUND OF INVENTION

Use of transdermal delivery systems for topical administration of an active ingredient are known in the art. Heretofore attempts have also been made to deliver transdermally small amounts of a xanthine composition containing caffeine for the treatment of a cosmetic condition as described in U.S. Patent Publication No. 2003/0152612 by Pugliese et al. Attempts also have been made to deliver transdermally relatively larger amounts of caffeine as a stimulant; however such attempts have met with limited success.

The caffeine-releasing transdermal patch of the present invention, overcomes prior art shortcomings and delivers caffeine from a specially formulated adhesive matrix at a rate that provides to the user a sustained base level of energy over an extended time period.

### SUMMARY OF INVENTION

A transdermal patch comprises a backing, a pressure-sensitive, hydrophobic acrylic adhesive matrix containing caffeine, and a protective release sheet over the adhesive matrix.

The adhesive matrix is a mixture of a solvent-based acrylic polymer having hydroxy groups and a solvent-based acrylic polymer without hydroxy groups. A xanthine composition comprising dried aqueous guarana seed extract, caffeine, and optionally cocoa, is distributed in the adhesive matrix, preferably in an amount in the range of about 5 to about 50 percent by weight, more preferably in the range of about 7 to about 45 percent by weight, based on the total weight of the adhesive matrix in the transdermal patch.

The transdermal patch is non-irritating and is useful for providing to a user a sustained energy boost over an extended time period.

Another optional constituent of the adhesive matrix is an arnica extract which can be present in an amount of up to 10 percent by weight, based on the total weight of the adhesive matrix.

### DESCRIPTION OF PREFERRED EMBODIMENTS

The pressure-sensitive acrylic adhesive matrix is hydrophobic and is made up by an acrylic copolymer mixture, one of the copolymers having hydroxy groups and the other copolymer having no hydroxy groups. Both copolymers contain carboxy groups, however.

A preferred hydroxy group containing acrylic copolymer is a self-crosslinking acrylic copolymer which comprises 2-hydroxyethyl acrylate, 2-ethylhexyl acrylate, acrylic acid and methyl methacrylate, together with cross-linking agents aluminum acetonate and tert-amyl peroxypivalate. One such hydroxy group containing acrylic copolymer is commercially available under designation Duro-Tak™ 87-2074 from Henkel Corporation, Bridgewater, N.J. 08807, U.S.A. as a colorless liquid.

A preferred acrylic copolymer without hydroxy groups comprises 2-ethylhexyl acrylate, acrylic acid, butyl acrylate and vinyl acetate. One such acrylic copolymer is commercially available under the designation Duro-Tak™ 87-2051 from Henkel Corporation, Bridgewater, N.J. 08807, U.S.A. as a colorless liquid.

In the adhesive matrix, the hydroxy group containing acrylic copolymer and the acrylic copolymer without hydroxy groups are present in a respective weight ratio in the range of about 1.5 to about 3.5, preferably a weight ratio of about 2 to about 3.

The terms “xanthine composition” and “xanthines” as used herein and in the appended claims include xanthine and its methylated derivatives commonly utilized as mild stimulants, i.e., caffeine, theobromine, theophylline, paraxanthine, and the like.

The xanthine composition is distributed in the acrylic adhesive matrix in an amount in the range of about 5 to about 50 weight percent, preferably about 7 to about 45 weight percent, based on the total weight of the matrix, and is constituted preferably by dry aqueous guarana (Paullinia cupana) seed extract fortified by caffeine as well as additional caffeine. Optionally, some of the caffeine can be present as a caffeine salt, e.g., caffeine citrate and the like pharmaceutically acceptable salt.

The caffeine content in the caffeine fortified guarana seed extract can vary, but usually is in the range of about 50 to about 58 weight percent, based on the dry weight of the fortified guarana seed extract. The caffeine fortified guarana seed extract can also contain theobromine as well as theophylline. The total amount of caffeine present in the adhesive matrix is in the range of about 6 to about 40 percent by weight, preferably in the range of about 7 to about 25 percent by weight, based on the total weight of the adhesive matrix.

A suitable caffeine-fortified guarana seed extract in powder form is commercially available under the designation Guarana SD SE 50% from Stryka Botanics, Hillsborough, N.J. 08844, U.S.A.

The transdermal patch embodying the present invention can also contain cocoa, an antioxidant, and can provide additional source of xanthines as well, particularly the methylated derivatives thereof. Cocoa can be present in the adhesive matrix of the patch in an amount in the range of about 0.5 to about 5 percent by weight, preferably about 1 to about 3 percent by weight, based on the total weight of the adhesive matrix.

As a further optional ingredient, the adhesive matrix can contain arnica (6-methoxykaempferol), usually as an extract of Arnica montana, in an amount of up to and including about 10 percent by weight, based on total weight of the adhesive matrix, for relief of pain. Suitable arnica extract is commercially available from Vitacost, Las Vegas, Nev. 89119.

The adhesive matrix also includes propylene glycol as a non-volatile solvent for the xanthines. Propylene glycol is present in an amount in the range of about 2 to about 10 weight percent, preferably about 5 to about 8 weight percent, based on total weight of adhesive matrix.

Polyvinylpyrrolidone in the adhesive matrix is optional and can serve as tackiness and peel strength modulator. Polyvinylpyrrolidone preferably has a weight average molecular weight in the range of about 2,000 to about 11,000 Daltons, more preferably about 7,000 to about 11,000 Daltons, and is present in an amount in the range of about 2 to about 5 weight percent, preferably about 2.3 to about 2.5 weight percent, based on total weight of the adhesive matrix. Suitable polyvinylpyrrolidones are commercially available under the designations Kollidon 12 PF and Kollidon 17 PF from BASF Corporation, Florham Park, N.J. 07932, U.S.A.

The backing for the acrylic adhesive matrix can be occlusive or semi-occlusive, and preferably has a moisture vapor transmission rate (MVTR) no more than about 1000 grams/m²/24 hours. A preferred foam backing is a relatively high vinyl acetate (VA) content ethylene-vinyl acetate (EVA)copolymer commercially available under the designation Volara® Type G from Sekisui-Voltek, Lawrence, Mass. 01843, U.S.A. Another preferred backing is a segment of a kinesiology tape which comprises a relatively thin, elastic, woven fabric coated with an acrylic adhesive on one side, or without the adhesive. The fabric can be made of natural fibers such as cotton and the like, synthetic fibers such as nylon, polyester, rayon, and the like, or a combination of natural and synthetic fibers. Kinesiology tape mimics the inherent elastic properties of human skin and enhances circulatory flow in the applied region as the wearer moves about. Illustrative kinesiology tapes are described in U.S. Pat. No. 5,861,348 to Kase and U.S. Patent Publication No. 2014/0102632 by Quinn.

Shape of the energy patch is determined by the configuration of the backing, and can vary. The energy patch can be round, oval, rectangular, trapezoid-like, an elongated strip of body-adhesive tape, and the like.

A protective release sheet or liner overlies the acrylic matrix prior to use of the patch, and is compatible with the acrylic adhesive matrix as well as with the xanthine composition dispersed therein. The release sheet or liner is composed of a material which protects the adhesive matrix prior to use, but is readily removed or separated from the adhesive matrix as the transdermal patch is prepared for use. Suitable are silicone or fluorocarbon coated films such as polyurethane films, polyester films, and the like. Suitable release sheets are commercially available from Fox River Associates, Geneva, Ill. 60134, U.S.A., and under the designation ScotchPak® 1022 from 3M Corporation, St. Paul, Minn. 55144, U.S.A.

The transdermal patches are prepared by first combining the dry ingredients, i.e., the caffeine-fortified guarana seed extract powder, caffeine, cocoa, etc., with a solvent such as ethyl acetate, and the like, with stirring to produce a substantially uniform dispersion which is then deposited on a release sheet or liner.

The acrylic adhesive matrix is prepared by combining the hydroxy-group containing acrylic copolymer with the aforesaid dry powder dispersion. The acrylic copolymer containing no hydroxy groups is thereafter added to the obtained dispersion with stirring until a homogenous mass is obtained. A solution of polyvinylpyrrolidone in propylene glycol is thereafter added to the homogenous mass with stirring to produce a caffeine-containing acrylic adhesive paste which is then deposited onto a release liner as a layer having a thickness of about 15 to 20 mils, and thereafter dried. Volatile solvent present in the deposited layer is removed by an air flow at a temperature in the range of about 50 to about 80° C. After solvent removal, the resulting acrylic adhesive layer containing the caffeine-fortified guarana seed extract has a thickness of at least about 3 mils, preferably about 3 to about 5 mils, is laminated to the backing, and cut to a desired patch size and configuration.

A single patch dosage unit can have a surface area in the range of about 10 to about 200 cm², preferably about 30 to about 60 cm².

### EXAMPLE 1

### Preparation of Energy Patch

Caffeine-fortified guarana seed extract in powder form and containing 57.9 weight percent caffeine (Guarana SD SE 50%) was weighed into a capped container. Ethylene acetate was added to the powder with stirring, and the resulting admixture was held at ambient room temperature for about 18 hours.

Propylene glycol and polyvinylpyrrolidone (Kollidon 12 PF) were combined in a separate container with stirring until complete dissolution.

A self-crosslinking acrylic copolymer comprising 2-hydroxyethyl acrylate, 2-ethylhexyl acrylate, acrylic acid, and methyl methacrylate (Duro-Tak™ 87-2074; 33% solids) was weighed into the container containing the fortified guarana seed extract powder and the resulting admixture was stirred for two hours until a homogeneous dispersion was obtained. Thereafter an acrylic copolymer of 2-ethylhexyl acrylate, acrylic acid, butyl acrylate and vinyl acetate (Duro-Tak™ 87-2051; 52% solids) was added to the homogeneous dispersion with stirring. Stirring was continued for one hour until a homogenous dispersion was obtained.

The dispersion containing both acrylic copolymers was then combined with the solution of polyvinylpyrrolidone in propylene glycol with stirring until a homogeneous adhesive paste was obtained. The obtained adhesive paste was kept for 12 hours at ambient room temperature until all entrained air had dissipated.

The adhesive paste thereafter was spread onto a siliconized surface of a polyurethane film release liner (Fox River Associates, Geneva, Ill.) to a thickness of about 13 mils and volatile solvent was removed therefrom by air flow at a temperature of about 75° C. After solvent removal the produced acrylic adhesive layer was about 8-10 mils thick and was laminated to an ethylene-vinyl acetate foam backing layer (Volara G Type) 0.020 inch thick. After lamination, the produced transdermal patch was cut and trimmed to produce a transdermal patch having a surface area of 51 cm².

Composition of the adhesive matrix during the foregoing manufacturing process is shown in Table 1 below.

TABLE 1
Patch Composition

| | Amount | | |
|---|---|---|---|
| Component | % w/w (wet) | % w/w (solids) | % w/w (dry) |
| Duro-Tak ™ 87-2074 | 52 | 17.2 | 41 |
| (33% solids) | | | |
| Duro-Tak ™ 87-2051 | 12.5 | 6.5 | 15.5 |
| (52% solids) | | | |
| Guarana SD SE 50% | 15 | — | 35.8 |
| Propylene glycol | 2.2 | — | 5.3 |
| Polyvinylpyrrolidone | 1.0 | — | 2.4 |
| Ethyl acetate | 17.3 | — | — |

Duro-Tak ™ 87-2074-to-Duro-Tak ™ 87-2051 weight ratio = 2.65

### EXAMPLE 2

### Caffeine Permeation

Permeation of caffeine from a transdermal patch prepared in accordance with Example 1, above, was determined in vitro using human cadaver skin in a Franz diffusion cell, pH 7.4 buffer. The observed results are compiled in Table 2, below.

TABLE 2
Permeation of Caffeine

| Time, | Cumulative Permeation, | |
|---|---|---|
| hrs. | mg/cm² | ±SD |
| 2 | 0.05 | 0.03 |
| 4 | 0.14 | 0.06 |
| 6 | 0.30 | 0.16 |
| 8 | 0.51 | 0.20 |
| 12 | 0.56 | 0.17 |
| 24 | 0.61 | 0.21 |

The foregoing data illustrate a continuous permeation of caffeine from the patch over a 24 hour time period; flux=0.61 mg/cm²/day.

### EXAMPLE 3

### Energy Patch with Added Caffeine and Cocoa

Guarana seed extract (Guarana SD SE 50%), caffeine granules (Knoll AG, Ludwigshafen (Rhein), Germany) and cacao powder (Healthworks, Scottsdale, Ariz. 85258; B004EKSZ4K) were combined in a mortar with pestle and reduced to a fine powder mix. The proportions by weight of the guarana seed extract, caffeine, and cacao were 2.5:6:1, respectively. The obtained fine powder mix was then stirred into ethyl acetate.

A hydroxy group containing acrylic polymer (Duro-Tak™ 87-2074) was combined with an acrylic polymer without hydroxy groups (Duro-Tak™ 87-2051) in a separate container with stirring to provide an adhesive matrix solution.

Propylene glycol and polyvinylpyrrolidone (Kollidon 12 PF) were combined in another container and stirred until a clear solution was obtained.

The adhesive matrix solution was combined with the obtained fine powder mix in ethyl acetate with vigorous stirring for two hours.

Thereafter the resulting fine powder admixture was combined with the propylene glycol-polyvinylpyrrolidone solution by slowly adding the solution, in divided portions, to the fine powder admixture with constant stirring for three days to produce a homogeneous adhesive paste which was maintained at ambient room temperature until all entrained air had dissipated and then used to produce transdermal patches as described in Example 1, above, having an acrylic adhesive layer about 10 mils in thickness prior to removal of ethyl acetate by air flow at about 75° F.

The backing material was an ethylene-vinyl acetate foam (Volara G Type, 0.020 inch thick).

Composition of the adhesive matrix during the foregoing manufacturing process is shown in Table 3, below.

TABLE 3
Energy Patch Composition

| | Amount | | |
|---|---|---|---|
| Component | % w/w (wet) | % w/w (solids) | % w/w (dry) |
| Duro-Tak ™ 87-2074 | 62.65 | 18.8 | 59.86 |
| (30% solids) | | | |
| Duro-Tak ™ 87-2051 | 12.4 | 6.45 | 20.64 |
| (52% solids) | | | |
| Guarana SD SE 50% | 0.78 | | 2.5 |
| Caffeine | 1.88 | | 6 |
| Cacao powder | 0.31 | | 1 |
| Propylene glycol | 2.42 | | 7.7 |
| Polyvinylpyrrolidone | 0.72 | | 2.3 |
| Ethyl acetate | 18.84 | | — |

Duro-Tak ™ 87-2074-to-Duro-Tak ™ 87-2051 weight ratio = 2.9

### EXAMPLE 4

### Caffeine Permeation

Permeation of caffeine from transdermal patches prepared in accordance with Example 3, above, was determined in vitro using human cadaver skin in a Franz diffusion cell, pH 7.4 buffer. The observed results are compiled in Table 4, below.

TABLE 4
Permeation of Caffeine

| Time, | Cumulative Permeation, | |
|---|---|---|
| hrs. | μg/cm² | ±SD |
| 1 | 251.04 | 17.62 |
| 2 | 320.81 | 19.56 |
| 4 | 420.66 | 30.78 |

### EXAMPLE 5

### Energy Tape

A layer of the adhesive composition shown in Table 3, above, was deposited on the adhesive side of kinesiology tape strips commercially available from Lumos Inc., Lindon, Utah 84042 (Black Tape—KT Tape Brand, 100% cotton fiber with elastic synthetic fiber core; Blue Tape—KT Tape Pro Brand, 100% rayon with elastic synthetic fiber core).

Permeation of caffeine was determined in vitro using a Franz diffusion cell, pH 7.4 buffer, and a nylon membrane having a pore size of 0.22 microns (Whatman 7402-002 White Nylon Membrane Filter, 25 mm diameter). The observed results are compiled in Table 5, below.

TABLE 5
Permeation of Caffeine from Kinesiology Tape

| | Cumulative Permeation | |
|---|---|---|
| Time, | Black Tape | Blue Tape |

| hrs. | μg/cm² | ±SD | μg/cm² | ±SD |
|---|---|---|---|---|
| 1 | 185.94 | 14.28 | 150.92 | 8.66 |
| 2 | 246.76 | 40.36 | 204.43 | 11.60 |
| 4 | 286.72 | 22.18 | 287.42 | 14.79 |

The foregoing description and the Examples are illustrative, and are not to be taken as limiting. Still other variants within the spirit and scope of this invention are possible and will readily present themselves to those skilled in the art.

We claim:

1. A transdermal patch comprising
a backing layer;
a pressure-sensitive acrylic adhesive matrix on the backing layer; and
a xanthine composition comprising guarana seed extract and caffeine in said matrix; and
a protective release sheet over the pressure-sensitive acrylic adhesive matrix;
the pressure-sensitive acrylic adhesive matrix comprising a mixture of an acrylic adhesive having hydroxy groups and an acrylic adhesive without hydroxy groups.

2. The transdermal patch in accordance with claim 1 wherein the xanthine composition is present in the matrix in an amount in the range of about 5 to about 50 percent by weight, based on the total weight of the adhesive matrix.

3. The transdermal patch in accordance with claim 1 wherein the acrylic adhesive having hydroxy groups and the acrylic adhesive without hydroxy groups are present in the matrix in a respective weight ratio in the range of about 0.5 to about 5.

4. The transdermal patch in accordance with claim 1 wherein the acrylic adhesive having hydroxy groups and the acrylic adhesive without hydroxy groups are present in the matrix in a respective weight ratio of about 2 to about 3.

5. The transdermal patch in accordance with claim 1 wherein the pressure-sensitive acrylic adhesive matrix further contains polyvinylpyrrolidone and propylene glycol.

6. The transdermal patch in accordance with claim 1 wherein the polyvinyl pyrrolidone is present in an amount in the range of about 2 to about 5 percent by weight of the total weight of the adhesive matrix and has a weight average molecular weight in the range of about 2,000 to about 11,000 Daltons, and propylene glycol is present in an amount in the range of about 2 to about 10 percent by weight of the total weight of the adhesive matrix.

7. The transdermal patch in accordance with claim 1 wherein the thickness of pressure-sensitive acrylic adhesive matrix is in the range of about 3 mils to about 5 mils.

8. The transdermal patch in accordance with claim 1 wherein the xanthine composition comprises a caffeine fortified guarana seed extract and caffeine.

9. The transdermal patch in accordance with claim 8 further containing cocoa.

10. The transdermal patch in accordance with claim 9 and containing about 0.5 to about 5 percent by weight cocoa, based on total weight of the adhesive matrix.

11. The transdermal patch in accordance with claim 8 wherein the caffeine is present in an amount in the range of about 6 to about 40 percent by weight of the total weight of the adhesive matrix, and cocoa is present in an amount in the range of about 1 to about 3 percent by weight, based on total weight of the adhesive matrix.

12. The transdermal patch in accordance with claim 1 wherein the acrylic adhesive having hydroxy groups is a self-crosslinking acrylic copolymer of 2-hydroxyethyl acrylate, 2-ethylhexyl acrylate, acrylic acid, and methyl methacrylate, containing aluminum acetonate and t-amyl peroxypivalate as cross-linking agents, and wherein the acrylic adhesive without hydroxy groups is a copolymer of 2-ethylhexyl acrylate, acrylic acid, butyl acrylate and vinyl acetate.

13. The transdermal patch in accordance with claim 1 wherein the release sheet is a silicone treated polyurethane film.

14. The transdermal patch in accordance with claim 1 wherein the foam backing layer is an ethylene-vinyl acetate foam.

15. The transdermal patch in accordance with claim 1 wherein the adhesive matrix is at least 3 mils thick.

16. A transdermal patch comprising
an occlusive foam backing layer;
a pressure-sensitive acrylic adhesive matrix on the backing layer;
guarana seed extract in said adhesive matrix;
caffeine in said adhesive matrix;
cocoa in said adhesive matrix; and
the adhesive matrix comprising a mixture of an acrylic adhesive having hydroxy groups and an acrylic adhesive without hydroxy groups.

17. The transdermal patch in accordance with claim 16 further including arnica in said adhesive matrix.

18. The transdermal patch in accordance with claim 16 containing about 2.5 percent by weight caffeine fortified guarana seed extract, about 6 percent by weight caffeine, and about 1 percent by weight cocoa, based on total weight of the adhesive matrix, and wherein the weight ratio of acrylic adhesive having hydroxy groups to acrylic adhesive without hydroxy groups is about 2.9.

19. The transdermal patch in accordance with claim 16 further containing up to about 10 percent by weight arnica extract, based on total weight of the adhesive matrix.

20. The transdermal patch in accordance with claim 1 further containing an arnica extract in an amount of up to about 10 percent by weight, based on total weight of the adhesive matrix.
